(19) United States
(12) Patent Application Publication
(10) Pub. No.: US 2019/0000335 A1
(43) Pub. Date: Jan. 3, 2019

(54) HEART MONITOR WITH DIAGNOSTIC DISPLAY AND ASSOCIATED METHODS

(71) Applicant: Medicomp, Inc., Melbourne, FL (US)

(72) Inventors: Tary Mann, Satellite Beach, FL (US); Anish Sobitharajan Mallika, Thiruvananthapuram (IN); Deepak Hariharan, Trivandrum (IN); Sreehari Krishnankutty, Trivandrum (IN); Arunlal Ramachandran Nair, Trivandrum (IN); Aruna Padmaja Sreekandan, Trivandrum (IN); Aparna Lalithakumari Amma Ramachandran Nair, Trivandrum (IN)

(73) Assignee: Medicomp, Inc, Melbourne, FL (US)

(21) Appl. No.: 16/126,641

(22) Filed: Sep. 10, 2018

Related U.S. Application Data
(63) Continuation-in-part of application No. 15/360,166, filed on Nov. 23, 2016, now abandoned.
(60) Provisional application No. 62/258,803, filed on Nov. 23, 2015.

(51) Int. Cl. A61B 5/044; A61B 5/00; A61B 5/046; A61B 5/0404; A61B 5/0468

(52) U.S. Cl. CPC A61B 5/044; A61B 5/0006; A61B 5/0468; A61B 5/0404; A61B 5/6822; A61B 5/046

(57) ABSTRACT

A heart monitor with diagnostic display including a plurality of electrodes structured to collect heart beat data from electrical pulses within a heart. The heart monitor also includes a processor adapted to receive the heart beat data from the plurality of electrodes and structured to connect with a long-range wireless network and a diagnostic display unit. The diagnostic display unit is structured to display biometric measurements including a first unit of time along a first axis and a second unit of time, smaller than the first unit of time, along a second axis. It also displays a gradient block having a primary characteristic indicative of a primary characteristic of a cardiac event detected by the processor in the heart beat data during a collection time and located at an intersection of the first axis and the second axis corresponding to a collection time of cardiac activity.

### RELATED APPLICATIONS

This continuation-in-part application claims the benefit under 35 U.S.C. § 119(e) of U.S. patent application Ser. No. 15/360,166 (Attorney Docket Number 612.00090) filed on Nov. 23, 2016, titled System and Method for Providing a Gradient Atrial Fibrillation Graph, which in turn claims the benefit of Provisional Patent Application Ser. No. 62/258.803 (Attorney Docket Number 612,00074), filed on Nov. 23, 2015, the entire contents of which are incorporated herein by reference.

### FIELD OF THE INVENTION

The present invention relates to the field of medical monitoring and, more specifically, to systems and methods for providing information related to patient cardiac events, particularly, atrial fibrillation, captured during electrocardiogram (ECG) monitoring.

### BACKGROUND

An individual's cardiac cycle consists of a number of stages under healthy physiological and anatomical conditions. Each recognizable component of the bioelectric signals generated by cardiac activity is labeled by convention. The entire cardiac cycle under normal conditions consists of a P-wave, a QRS complex, a T-wave and, in some cases, a U-wave. Each of these components represents a different stage in the cardiac cycle. The P-wave is representative of normal atrial depolarization, or contraction. When functioning properly, the atria of the heart receive blood either form the vena cava or from the pulmonary vein and pump it into their respective ventricles. Under abnormal conditions, specifically atrial fibrillation, the otherwise characteristic P-wave is no longer observable via monitoring equipment. Instead, an almost sinusoidal waveform is present between the T and Q waves. This abnormal waveform is indicative of a state of inefficiency of the atrial pumping mechanism, which causes abnormal flow characteristics in blood within the atria. The natural biological response of blood constituents to such irregular flow characteristics is to initiate the clotting factor cascade, which causes dots to form within the heart. Due to this fact, atrial fibrillation is conducive to producing blood clots that could lead to stroke, embolism, and other serious medical conditions: therefore, detection and total evaluation of this condition is extremely important.

A patient's cardiac cycle can be observed with the use of any standard electrocardiography (ECG) equipment. Traditionally, a patient would be required to go to his or her physician's office in order to undergo such testing. However, many advances in the field have avowed for these monitoring systems to be made ambulatory.

Conventional systems used to monitor atrial fibrillation burden on a patient involve the implementation of a single triggering threshold. These currently used systems will only record atrial fibrillation burden wherein a given atrial fibrillation event lasts for a minimum of a certain specified duration.

These monitoring systems and the data they collect and analyze are used by physicians to develop and modify treatments for atrial fibrillation and other cardiac conditions, a medical practice commonly known as titration. The timing and specific duration of atrial fibrillation events play a significant role in the development of these treatment procedures.

Current methods of analysis of atrial fibrillation data are limited by the implemented systems mentioned above. Conventional systems and methods are only able to provide generalized data limited to overall duration of atrial fibrillation burden events which would be qualified under the specified threshold. This shortcoming leaves the recipients of the data with only a crude understanding of a given patient's condition.

This background information is provided to reveal information believed by the applicant to be of possible relevance to the present invention. No admission is necessarily intended, nor should be construed, that any of the preceding information constitutes prior art against the present invention.

### SUMMARY OF THE INVENTION

A heart monitor with diagnostic display including a plurality of electrodes structured to collect heart beat data from electrical pulses within a heart. The heart monitor also includes a processor structured to receive the heart beat data from the plurality of electrodes and to connect with a long-range wireless network and a diagnostic display unit. The diagnostic display unit is structured to display biometric measurements including a first unit of time along a first axis and a second unit of time, smaller than the first unit of time, along a second axis. It also displays a gradient block having a primary characteristic indicative of a primary characteristic of a cardiac event detected by the processor in the heart beat data during a collection time and located at an intersection of the first axis and the second axis corresponding to a collection time of cardiac activity.

In some embodiments the second axis may include a dynamically adjustable scale and the gradient block may have a secondary characteristic indicative of a secondary characteristic of the cardiac event. Furthermore, each of the plurality of gradient blocks may present only if a characteristic of the cardiac activity is above a threshold level.

In some embodiments the collection time may have a duration of 30 minutes and the primary characteristic of the gradient block may be shade. A darker shade may correspond to a primary characteristic of a cardiac event of greater medical concern than a lighter shade. Furthermore, a primary characteristic of the cardiac event may be frequency. The cardiac event may include atrial fibrillation. Also, the first unit of time may include a calendar day whereby the first axis is the major axis, the second unit of time includes the time of day, and the second axis includes the minor axis.

One embodiment of the invention may include a method of monitoring heart beat activity including utilizing a heart rate monitor to record electrical pulses output by a patient's heart, the heart rate monitor may include a plurality of electrodes. It may involve utilizing a portable computing device configured to receive data from the heart rate monitor and transmitting the data via a long-range wireless network. It may utilize a receiving center to receive the data transmitted via the long-range wireless network and may transmit the data received by the receiving center to at least one technician for the identification of anomalous heart beat data. Furthermore, the method may include submitting the anomalous heart beat data into interface software and utilizing the interface software to transmit the anomalous heart beat data to display software. The display software may be used to display, on a diagnostic display unit, the anomalous heart beat data. The diagnostic display unit may be structured to display biometric measurements to a healthcare professional whereby the diagnostic display unit may be structured to display a first unit of time along a first axis and a second unit of time, smaller than the first unit of time, along a second axis. It may display a plurality of gradient blocks each having a primary characteristic indicative of a primary characteristic of a cardiac event detected during a collection time and located at respective intersections of the first axis. It may display a second axis corresponding to respective collection times of cardiac activity. In some embodiments each of the plurality of gradient blocks may present only if a characteristic of the cardiac activity is above a threshold level.

In one embodiment the second axis may include a dynamically adjustable scale. Furthermore, the plurality of gradient blocks may include a secondary characteristic indicative of a secondary characteristic of the cardiac event. The collection time may include a duration of 30 minutes. The primary characteristic of the plurality of gradient blocks may be shade and a primary characteristic of the cardiac event may be frequency. A darker shade may correspond to a greater frequency than a lighter shade and a cardiac event may include atrial fibrillation. Additionally, a first unit of time may include a calendar day whereby the first axis may include the major axis and the second unit of time may include the time of day. The second axis may include the minor axis.

Another embodiment of the invention may include a method of monitoring heart beat activity including utilizing a heart monitor. The heart monitor may include a plurality of electrodes structured to collect heart beat data from electrical pulses within a heart. It may include a processor adapted to receive the heart beat data from the plurality of electrodes and to connect with a long-range wireless network. It may include a diagnostic display unit. The diagnostic display unit may be structured to display biometric measurements including a first unit of time along a first axis and a second unit of time, smaller than the first unit of time, along a second axis. It may also display a gradient block having a primary characteristic indicative of a primary characteristic of a cardiac event detected by the processor in the heart beat data during a collection time. The gradient block may be located at an intersection of the first axis and the second axis and may correspond to a collection time of cardiac activity.

The method may utilize the processor to transmit the data via a long-range wireless network. It may utilize a receiving center to receive the data transmitted via a long-range wireless network and may transmit the data from the receiving center to at least one technician for the identification of anomalous heart beat data. The anomalous heart beat data may be submitted into interface software that may transmit the anomalous heart beat data to display software within the pendant. The display software may display on the pendant the anomalous heart beat data and biometric measurements to a healthcare professional. In this embodiment each of the plurality of gradient blocks may present only if a characteristic of the cardiac activity is above a threshold level.

### BRIEF DESCRIPTION OF THE DRAWINGS

FIG. 1 is a graphical representation of measured cardiac information in accordance with an embodiment of the present invention.

FIG. 2 is a zoomed out graphical representation of the graphical representation depicted in FIG. 1.

FIG. 3 is a zoomed in graphical representation of the graphical representation depicted in FIG. 1.

FIG. 4 is a flowchart describing a method according to the present invention to create the graphical representation of FIG. 1.

FIG. 5 is a flowchart describing a method to perform step 36 of FIG. 4 in accordance with an embodiment of the present invention.

FIG. 6 is a block diagram embodiment of a method of monitoring a patient's heart beat activity with a portable computing device separate from a pendant.

FIG. 7 is a flowchart embodiment of a method of monitoring a patient's heart beat activity with a portable computing device and display monitor built into a pendant.

FIG. 8 is a perspective view embodiment of a pendant with built in display monitor and electrodes.

FIG. 9 is a perspective view embodiment of a pendant with built in display monitor and electrodes.

FIG. 10 is a perspective view embodiment of a pendant with built in display monitor and electrodes.

FIG. 11 is a flow chart illustrating an overview of one embodiment of how information may be retrieved to build a heart monitor graph.

FIGS. 12a-12f are flowcharts illustrating an embodiment of a process of budding a heart monitor graph.

FIG. 13 is a flowchart illustrating one embodiment of how strip representations of heart beat activity are built.

### DETAILED DESCRIPTION OF THE INVENTION

The present invention will now be described more fully hereinafter with reference to the accompanying drawings, in which preferred embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. Those of ordinary skill in the art realize that the following descriptions of the embodiments of the present invention are illustrative and are not intended to be limiting in any way. Other embodiments of the present invention will readily suggest themselves to such skilled persons having the benefit of this disclosure. Like numbers refer to like dements throughout.

Although the following detailed description contains many specifics for the purposes of illustration, anyone of ordinary skill in the art will appreciate that many variations and alterations to the following details are within the scope of the invention. Accordingly, the following embodiments of the invention are set forth without any loss of generality to, and without imposing limitations upon, the claimed invention.

In this detailed description of the present invention, a person skilled in the art should note that directional terms, such as “above,” “below,” “upper,” “lower,” and other like terms are used for the convenience of the reader in reference to the drawings. Also, a person skilled in the art should notice this description may contain other terminology to convey position, orientation, and direction without departing from the principles of the present invention.

Furthermore, in this detailed description, a person skilled in the art should note that quantitative qualifying terms such as “generally,” “substantially,” “mostly,” and other terms are used, in general, to mean that the referred to object, characteristic, or quality constitutes a majority of the subject of the reference. The meaning of any of these terms is dependent upon the context within which it is used, and the meaning may be expressly modified.

An embodiment of the invention, as shown and described by the various figures and accompanying text, provides a novel means of presenting the frequency, intensity, and type of cardiac events as they occur over a period of time. This method may be particularly beneficial in presenting information related to anomalous cardiac events. By way of example, and not as a limitation, anomalous cardiac events may include atrial fibrillation, tachycardia, atrial flutter, bradycardia, ventricular fibrillation, or the like.

A patient may wear a heart monitoring device. The heart monitoring device may have one or more sensors for measuring a patient's heart activity. The heart monitoring device may record information associated with electrical signals from the heart. The heart monitoring device may store the measured cardiac information locally or provide it to an external device for storage. The cardiac information may be analyzed and classified. The classification may be made based on one or more characteristics present in the cardiac information. Classifications of the information may correspond to one or more cardiac events, which may be anomalous.

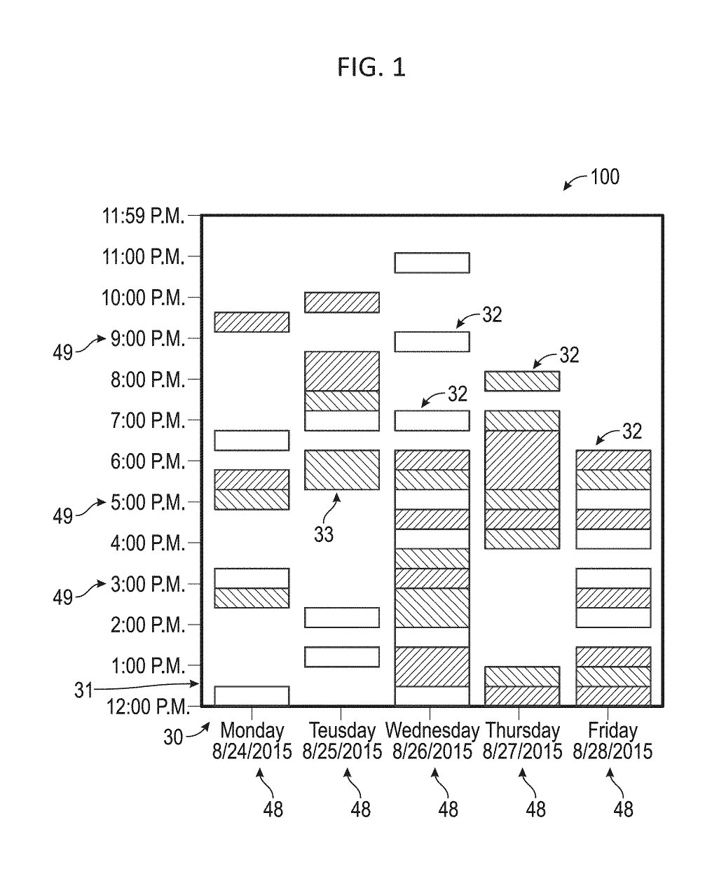

This system 100 may involve the creation or implementation of a graphical representation, which, by way of example, and not as a limitation, may be a table, graph, or the like, to graphically depict the results of the analysis of the cardiac information, as depicted in FIG. 1. The data that populates the graph may be sensed by a physiological signal monitoring system as disclosed in PCT Publication No. WO 2016/168315, which is hereby incorporated by reference in its entirety.

In one embodiment, the major axis 30 may be a first axis and may indicate the calendar day 48, which may be a first unit of time. The minor axis 31 may be a second axis and may indicate the time of day 49, which may be a second unit of time. Each gradient block 32 at the intersection points of the minor axis 31 and the major axis 30 may present information regarding one or more aspect of cardiac activity occurring at that time, hi one embodiment, the gradient block 32 may present information related to the percentage or absolute number of cardiac cycles that were classified as anomalous during the time period represented by the gradient block 32. The gradient block 32 may be shaded with differing intensity to correspond with the level of detected anomalous cardiac activity. By way of example, and not as a limitation, a relatively darker shaded gradient block 32 may be located at intersections during which more severe or more frequent anomalous cardiac events occur. If no anomalous activity occurs during a time period, no gradient block 32 may be presented at the corresponding time period on the graph.

A plurality of shaded gradient blocks 32 may be selected to represent the frequency, severity, or type of cardiac events. Color, gradient level, fill pattern, or the like of the gradient block 32 may relate to cardiac information recorded at the time corresponding to the location of the gradient block 32. In one embodiment, gradient level of the gradient block 32 may relate to frequency, severity, or type of the associated cardiac event. In another embodiment, color may relate to severity, frequency, or type of cardiac event. In yet another embodiment, fill pattern may relate to severity, frequency, or type of cardiac event. A primary characteristic of the gradient block 32, which may be color, gradient level, fill pattern, or the like, may relate to a primary characteristic of the cardiac event, which may be frequency, severity, or type. A secondary characteristic of the gradient block 32, which may be color, gradient level, fill pattern, or the like, and may be different from the primary characteristic of the gradient block 32, may relate to a secondary characteristic of the cardiac event, which may be frequency, severity, or type and may be different from the primary characteristic of the cardiac event. A tertiary characteristic of the gradient block 32, which may be color, gradient level, fill pattern, or the like, and may be different from the primary and secondary characteristics of the gradient block 32, may relate to a tertiary characteristic of the cardiac event, which may be frequency, severity, or type and may be different from the primary and secondary characteristics of the cardiac event.

By way of example, and not as a limitation, darker gradient blocks 32 may represent more frequent or more severe cardiac events. In one embodiment, only frequency of a single type of cardiac event may be depicted on the graphical representation. In such an embodiment, no gradient block 32 may be displayed if the cardiac event is not detected during the corresponding time period. There may be gradient blocks 32 of increasing darkness presented to correspond with detection of cardiac events within a range. By way of example, and not as a limitation, if 1-25 cardiac events are detected, the corresponding gradient block 32 may be lighter, if 26-50 cardiac events are detected, the corresponding gradient block 32 may be light, but darker than the lighter gradient block 32, if 51-75 cardiac events are detected, the corresponding gradient block 32 may be dark, and if more than 75 cardiac events are detected, the corresponding gradient block 35 may be darker. In another embodiment, only the severity of a single type of cardiac event may be depicted on the graphical representation. In yet another embodiment, either frequency or severity of a single type of cardiac event may be depicted and represented as a gradient level of the gradient block 32 while the other factor (frequency or severity) may be depicted and represented as a color or fill pattern of the gradient block 32. Those skilled in the art will appreciate that these characteristics are provided for exemplary purposes and other combinations of visual depictions are contemplated and contained within this disclosure.

A plurality of gradient blocks 32 may be disposed on the graphical representation. Each gradient block 32 may depict information obtained from the patient at the time corresponding to the intersection of the major axis 30 with the minor axis 31 at the location at which the gradient block 32 is located. Each gradient block 32 may represent information related to cardiac information obtained from a patient over an interval of time. By way of example, and not as a limitation, each gradient block 32 may represent one minute, two minutes, five minutes, 20 minutes, 30 minutes, one hour, or the like.

By way of example, and not as a limitation, each gradient block may represent a 30 minute period and be located at the corresponding intersection of the minor axis 31 and major axis 30 during which the cardiac information was measured. In embodiments in which the graphical representation is used only to report the occurrence of atrial fibrillation, the number of atrial fibrillation events detected between 6:00 am. and 6:30 am. on the second day 48 of monitoring may be calculated and displayed at location 33 on the graphical representation. In such an embodiment, the greater the number of atrial fibrillation events recorded during that time, the darker the gradient block 32 displayed at location 33 would be. Correspondingly, the fewer the number of atrial fibrillation events recorded during that time, the lighter the gradient block 32 displayed at location 33 would be. Similarly, the percentage of detected heartbeats that are classified as atrial fibrillation may be calculated and displayed. If less than a lower threshold number, or threshold severity, of cardiac events are detected, no gradient block 32 may be present on the graphical representation at the corresponding intersection of the major axis 30 and minor axis 31.

By way of example, and not as a limitation, type of cardiac event, frequency of cardiac event, and severity of cardiac event may all be graphically represented using gradient blocks 32. In such an embodiment, the fill pattern of each gradient block 32 may correspond to a different type of cardiac event. The color of each gradient block 32 may correspond to the severity of cardiac events. The gradient level of each gradient block 32 may correspond to the frequency of cardiac events. A legend, or key, may be included with each graphical representation to indicate the cardiac feature related to each visual element of the graphical representation. Those skilled in the art will appreciate that different combinations of visual features and cardiac characteristics are contemplated.

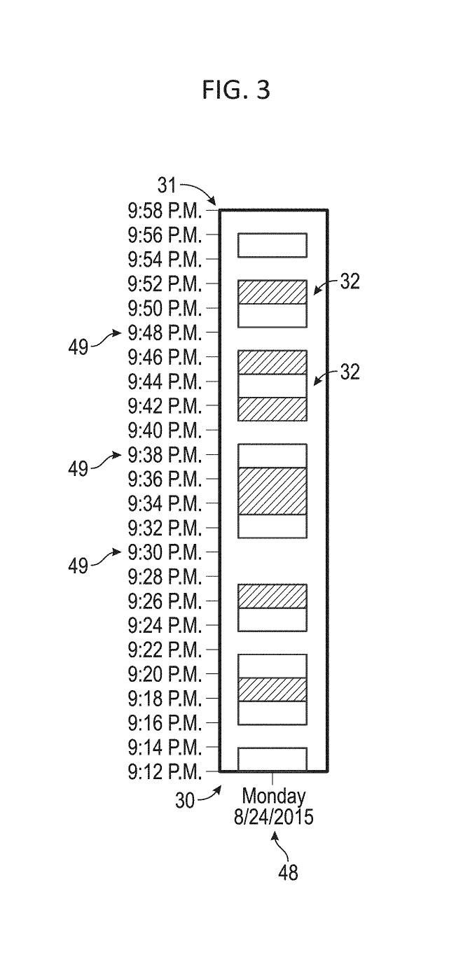

In one embodiment, a viewer of the graphical representation may adjust the timescale being viewed. FIG. 1 depicts information related to cardiac activity measured from a second unit of time 49 12:00 p.m. to 11:59 p.m. during a first unit of time 48 Monday, Aug. 24, 2015 through Friday, Aug. 28, 2015. FIG. 3 depicts a zoomed in graphical representation of FIG. 1. Specifically, FIG. 3 depicts information related to cardiac activity measured from a second unit of time 49 9:12-10:00 p.m. during a first unit of time 48 Tuesday, Aug. 25, 2015. In such an embodiment, zooming into the graphical representation may result in viewing a shorter duration of time. Accordingly, each gradient block 32 may represent a smaller duration of time and, therefore, a smaller absolute number of cardiac events. The ability to zoom into and out of the timescale may be referred to as having a dynamically adjustable scale.

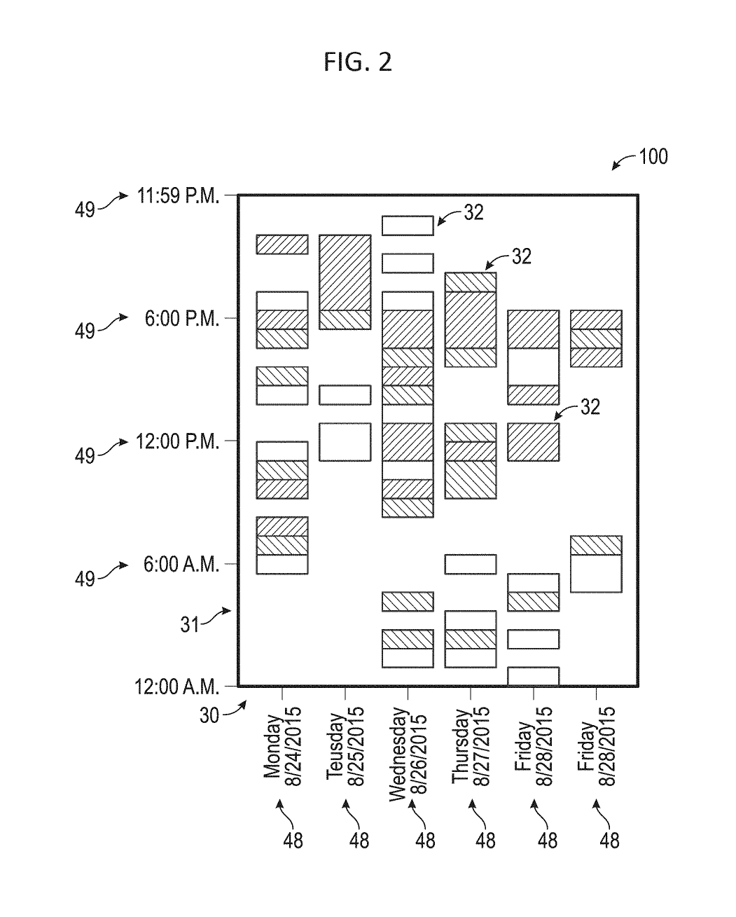

FIG. 2 depicts a zoomed out graphical representation of FIG. 1. Specifically, FIG. 2 depicts information related to cardiac activity measured from a second unit of time 49 12:00 a.m. to 11:59 p.m. during a first unit of time 48 Monday, Aug. 24, 2015 through Sunday, Aug. 30, 2015. Zooming out of the graphical representation may result in viewing a longer duration of time. Accordingly, each gradient block 32 may represent a larger duration of time and, therefore, a larger absolute number of cardiac events.

When changing the timescale being viewed, the gradient level, fill pattern, or color of the gradient block 32 may be altered to reflect the cardiac activity occurring during the time period associated with the gradient block 32.

The graphical representation may include information related to the total percentage of time the patient experiencing anomalous cardiac events. The graphical representation may be configured to depict the percentage of time the patient spent in a specified anomalous cardiac event type during a specified duration, during the entire monitoring duration, during a configurable range of time, or the like. The percentage of time a patient spends in a cardiac event may be displayed on the graphical representation displaying the time period for which the percentage is calculated.

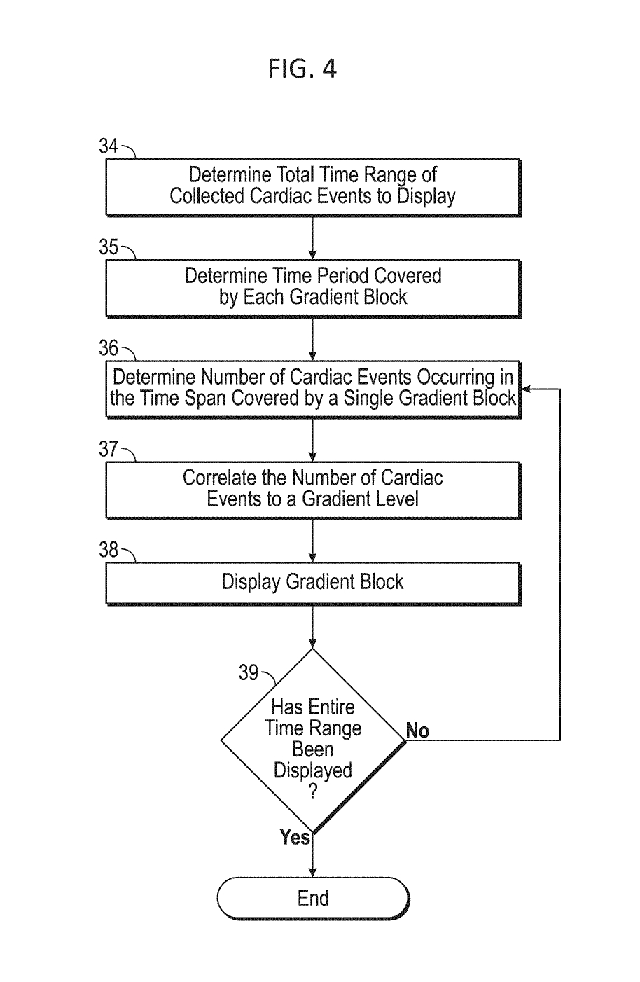

FIG. 4 depicts an embodiment of the inventive method for creating a graphical representation depicting the frequency of cardiac events. The desired time range of collected cardiac events to be displayed must be determined 34. This may be determined by a system default value, a user selected value, or the like. The time period covered by each gradient block must be determined 35. This may be calculated based on the total time range selected in step 34. The time period covered by each gradient block may be determined by a system default value, a user selected value, or the like. The default time period covered by each gradient block may correspond to the total time range to be displayed. The number of target cardiac events occurring in the time span covered by each gradient block must be determined 36. Starting from the beginning of the total time range to be displayed, through the end of the time period covered by a single gradient block, the total number of relevant cardiac events must be counted. The number of detected cardiac events may be correlated to a gradient level. A gradient block with the corresponding gradient level may be displayed 38. The method must determine whether or not the entire time range has been displayed in gradient blocks 39. If the entire time range has been displayed, the method is complete. If there is a portion of the time range left to be displayed, the number of cardiac events occurring in the time span covered by the next single gradient block may be determined 36.

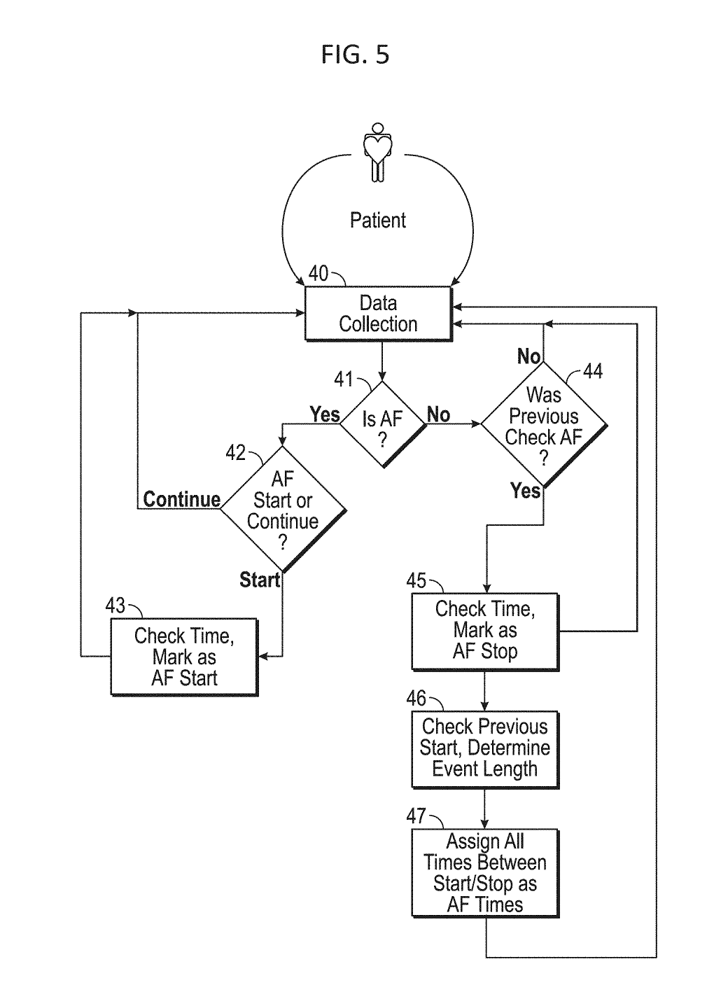

FIG. 5 depicts an embodiment of the inventive method that may be utilized to perform at least a portion of step 36 of FIG. 4. Data is collected from a patient 40. This data may be collected using a biophysical sensor. The sensor may be an electrode. The electrode may measure electrical heart activity. The collected information is analyzed to determine if the patient is experiencing atrial fibrillation 41. If the patient is experiencing atrial fibrillation, the system 100 determines whether or not this is the beginning occurrence of atrial fibrillation or if the previously measured patient waveform was atrial fibrillation 42. In instances in which the measured atrial fibrillation is the beginning of an occurrence of atrial fibrillation, the time is marked as an atrial fibrillation start time 43. In instances in which the measured atrial fibrillation is a continuing occurrence of atrial fibrillation, the system 100 continues to monitor the patient. When atrial fibrillation is no longer detected by the system 100, the system 100 determines whether the previously detected waveform was atrial fibrillation 44. In instances in which the absence of atrial fibrillation occurs after an instance of atrial fibrillation, the time is marked as an atrial fibrillation stop time 45. The length of the atrial fibrillation event is determined utilizing the atrial fibrillation start and end times 46. All time between the start and end times is classified as atrial fibrillation time 47 and the system 100 continues to collect patient data 40. In instances in which the absence of atrial fibrillation occurs immediately after a previous absence of atrial fibrillation, the system 100 continues to collect patient data 40.

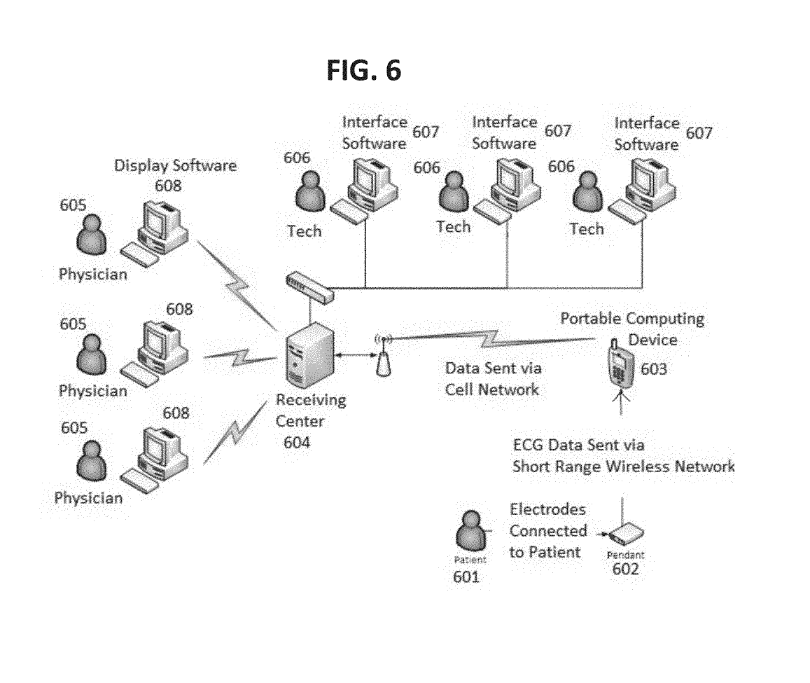

FIG. 6 illustrates one embodiment of monitoring a patient's heart beat activity whereby a portable computing device 603 is separate from a pendant 602, which may be worn by a patient 601. In this embodiment, a patient 601 may be connected to a pendant 602 via electrodes located on the pendant 602. The pendant 602 may monitor electrical pulses generated by the patient's 601 heart. The pendant 602 may then submit data regarding the frequency of pulses via a short-range wireless network to the portable computing device 603. The portable computing device 603 may then forward the heart beat data to a centralized receiving center 604 via a long-range wireless network. The receiving center 604 may then forward the heart beat data to at least one technician 606 that may flag anomalous heart beat data to be entered into interface software 607. In some embodiments, the interface software 607 may be named Beacon. The interface software 607 may interface with display software 608 by way of the receiving center 604. In some embodiments the display software 608 may be named AAW. The display software 607 may then formulate the graphs previously described. The display software 607 may display the anomalous heart beat data to a physician 605 located either remotely from the patient 601 or on a display unit at the patient's 601 facility. The display software 607 may allow for the physician 605 to formulate a diagnosis.

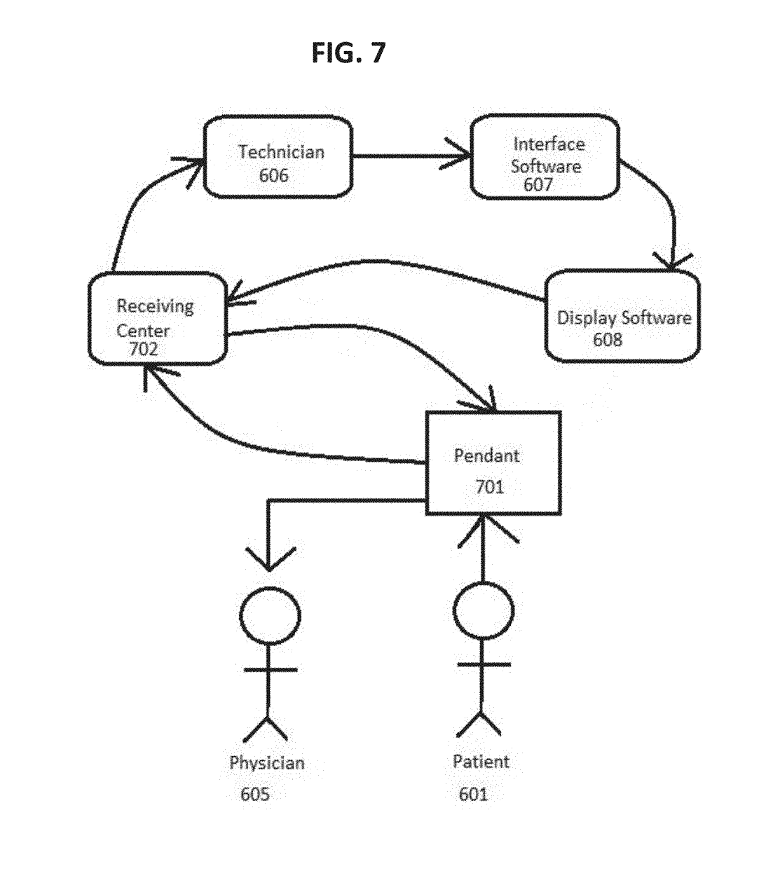

FIG. 7 illustrates another embodiment whereby the pendant 602 encompasses the portable computing device 603. Similar to FIG. 6, the patient 601 may be connected to the pendant 602 via electrodes located on the pendant 602. The pendant 602 may monitor electrical pulses generated by the patient's 601 heart. The pendant 602 may then submit the heart beat data to the receiving center 702 via long-range wireless network. The receiving center 702 may then forward the heart beat data to at least one technician 606 that may flag anomalous heart beat data to be entered into the interface software 607. The interface software 607 may interface with display software 608 by way of the receiving center 604. The display software 607 may then formulate the graphs previously described. The display software 607 may display the anomalous heart beat data by way of graphs displayed on the pendant 701. In this embodiment, the physician 605 may view the heart beat data on a graph displayed on the pendant 701 connected to the patient 601.

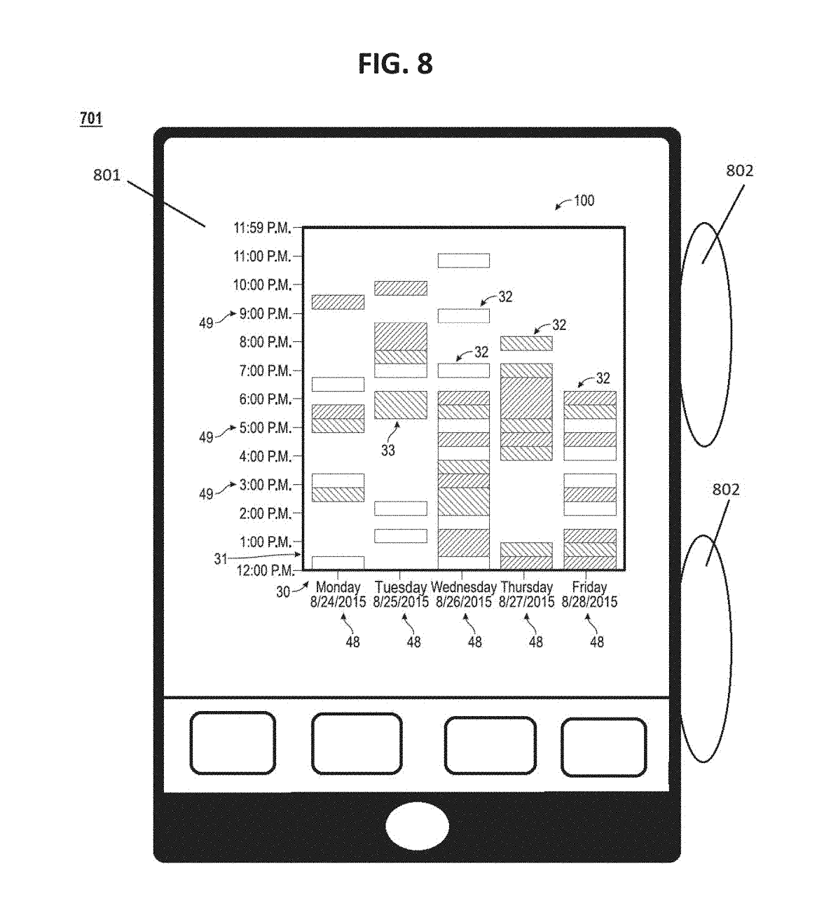

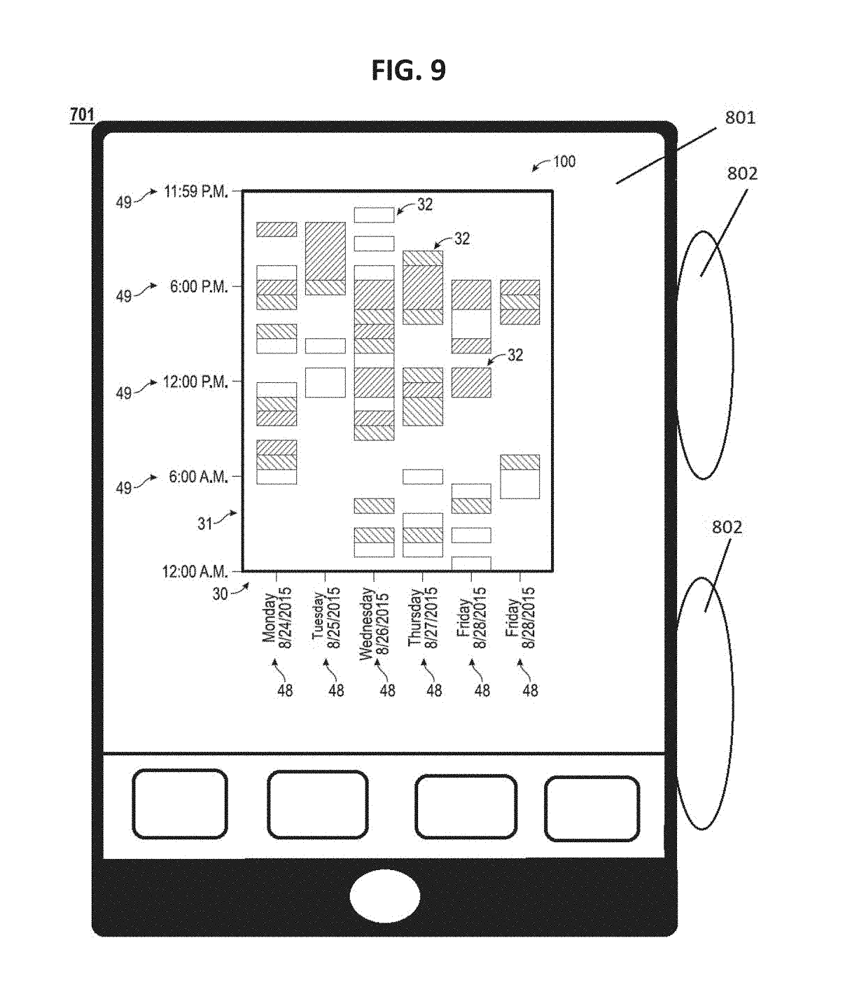

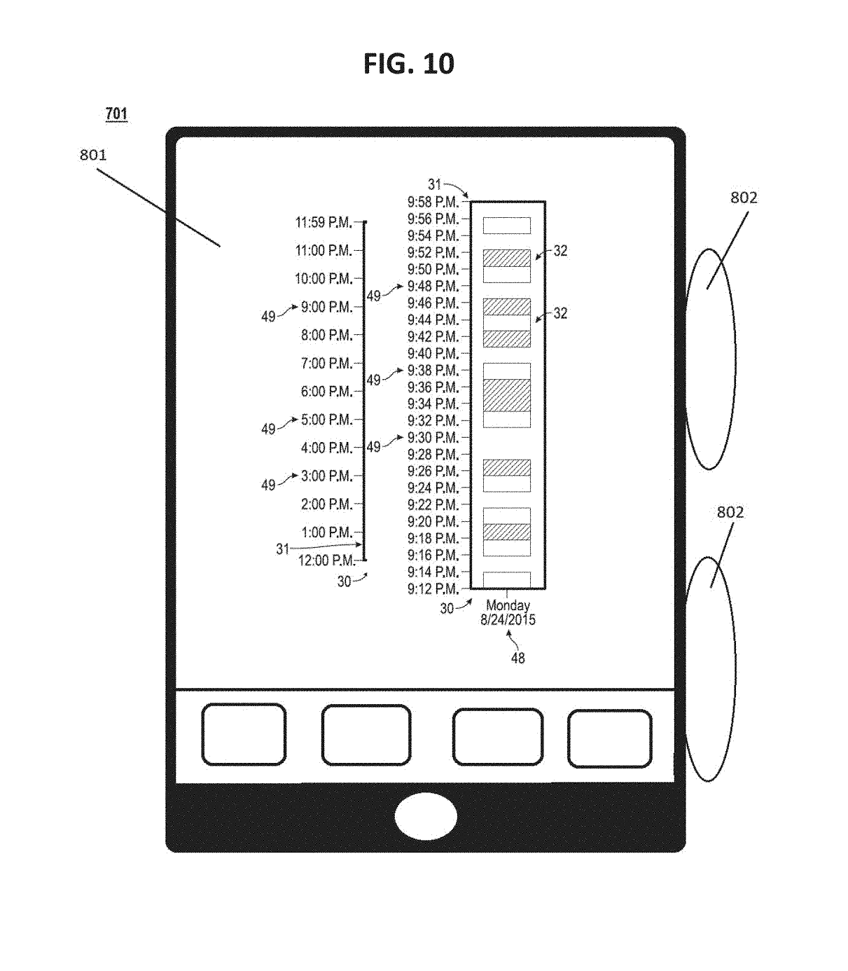

FIGS. 8-10 illustrate a heart monitor pendant 701 with a display unit 801 within the pendant 701. As illustrated, the pendant 701 may include a diagnostic display unit 801 structured to display biometric measurements in charts described hereinabove. The pendant 701 may electrically connect to the patient 601 via a plurality of electrodes 802 located thereon. The plurality of electrodes 802 may be structured to collect heart beat data from electrical pulses within a patient's 601 heart. The pendant 701 may include a portable computing device (not shown) structured to connect with a short-range wireless network and a long-range wireless network. In some embodiments the portable computing device may be separate from the pendant 701 and in some embodiments it may be included within the pendant 701. The pendant 701 may also include a processor to coordinate and process the pendant's 701 tasks.

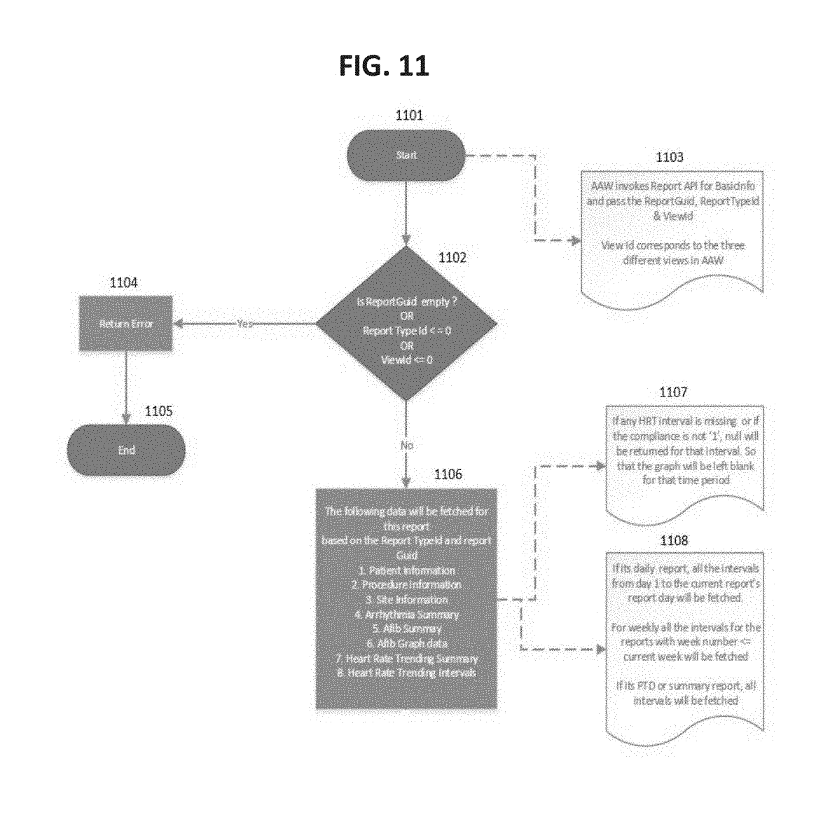

FIG. 11 gives an overview of what information may be used and retrieved for the display of a heart monitor graph. Block 1103 indicates that the AAW software, also referenced as the display software 607, may initiate three different views of heart beat data. Blocks 1102, 1104 and 1105 represent error handling for the display software 607. Block 1106 indicates that data to be fetched for the display include patient information, procedure information, site information, arrhythmia summary, atrial fibrillation (“afib”) summary, atrial fibrillation (“afib”) graph data, heart rate trending summary and heart rate trending intervals. Block 1107 notes that if any heart interval is missing or if the compliance is not equal to 1, a null value will be returned for that interval. In that instance, the graph will be left blank. Block 1108 notes that if the display is a daily report, all the intervals from day 1 to the current report's report day will be fetched. For weekly reports and display, all the intervals for the reports with the week number corresponding to the current week will be fetched. If it is a PTD or summary report, all intervals will be fetched.

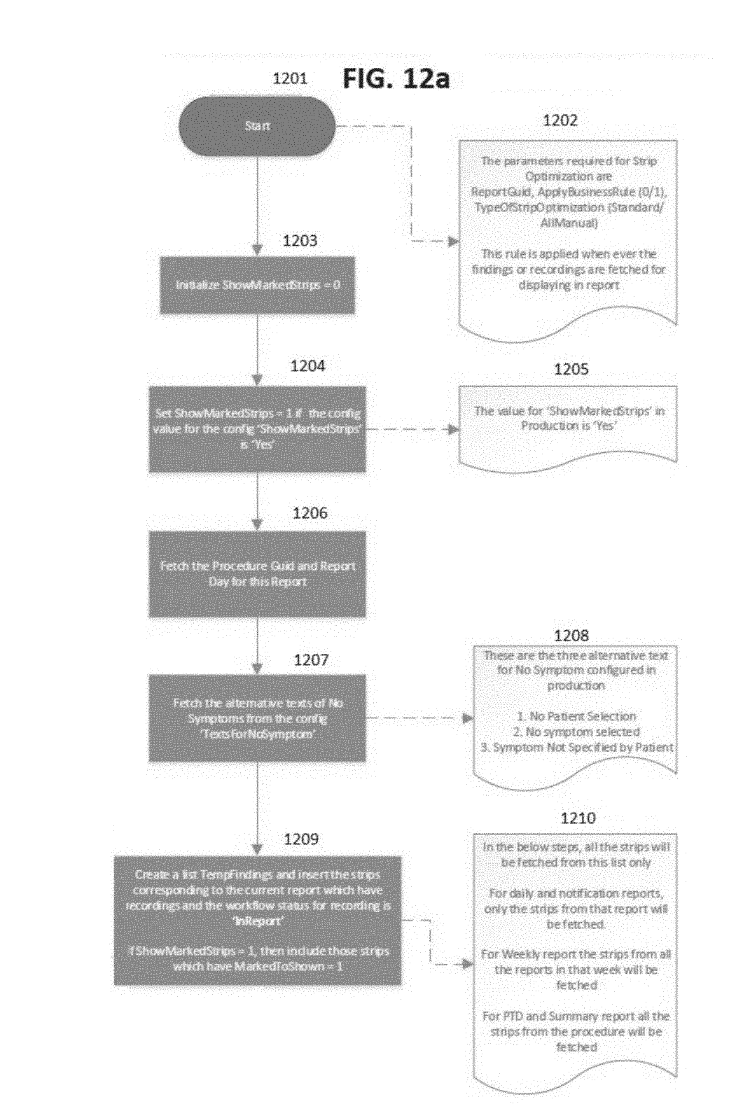

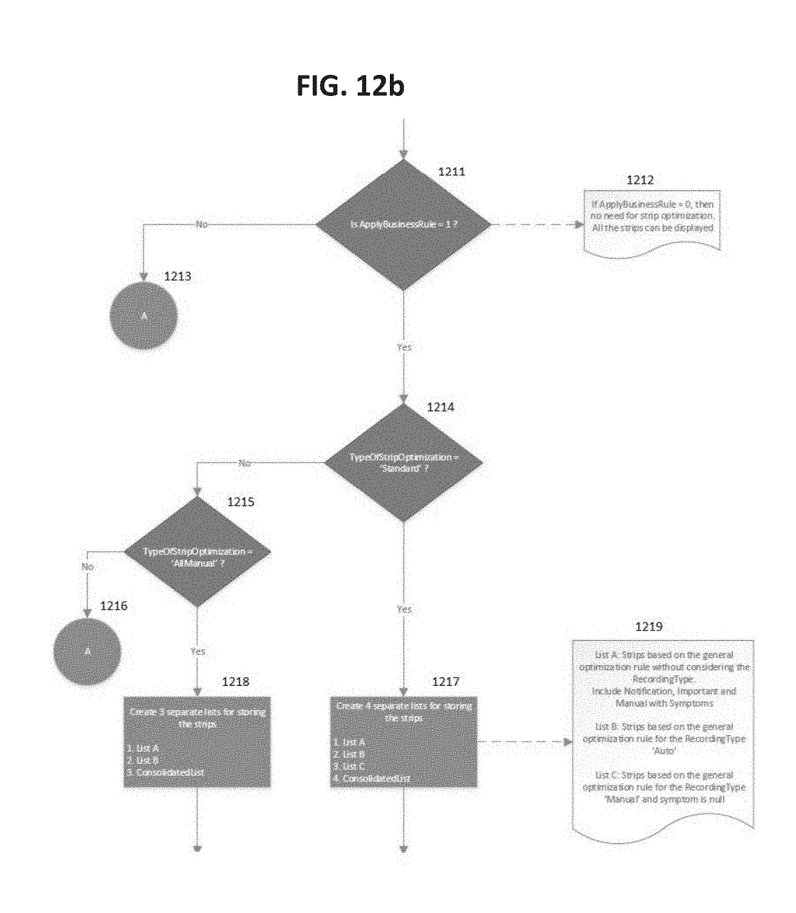

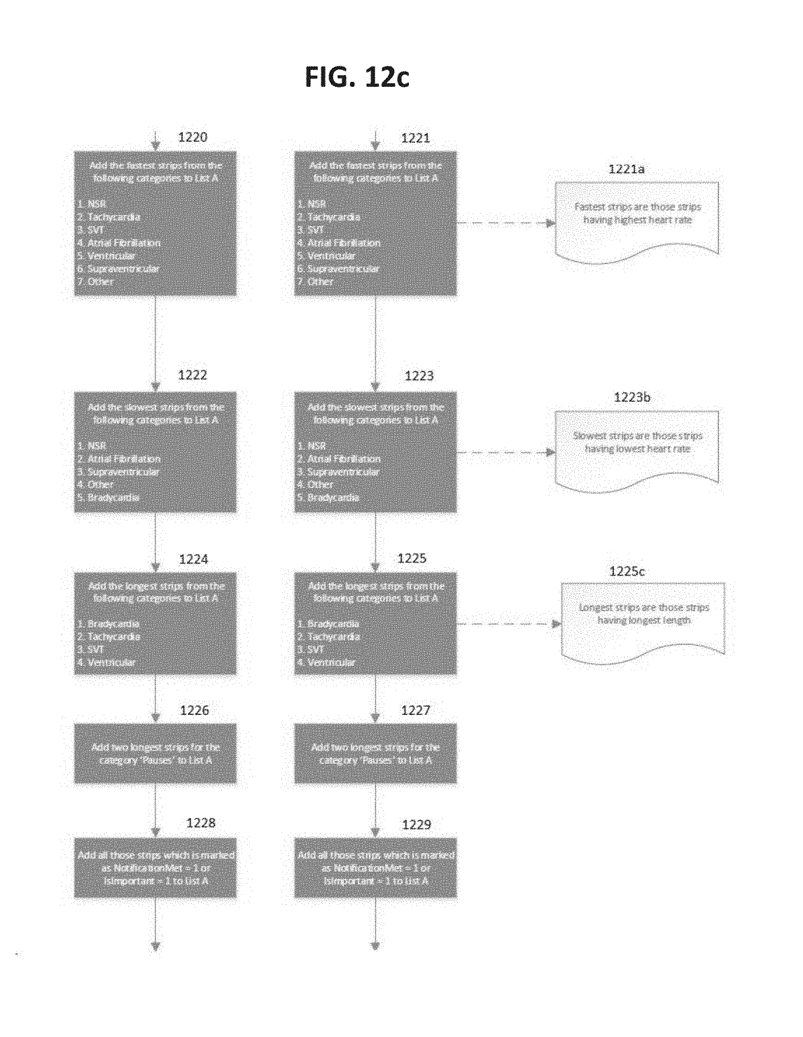

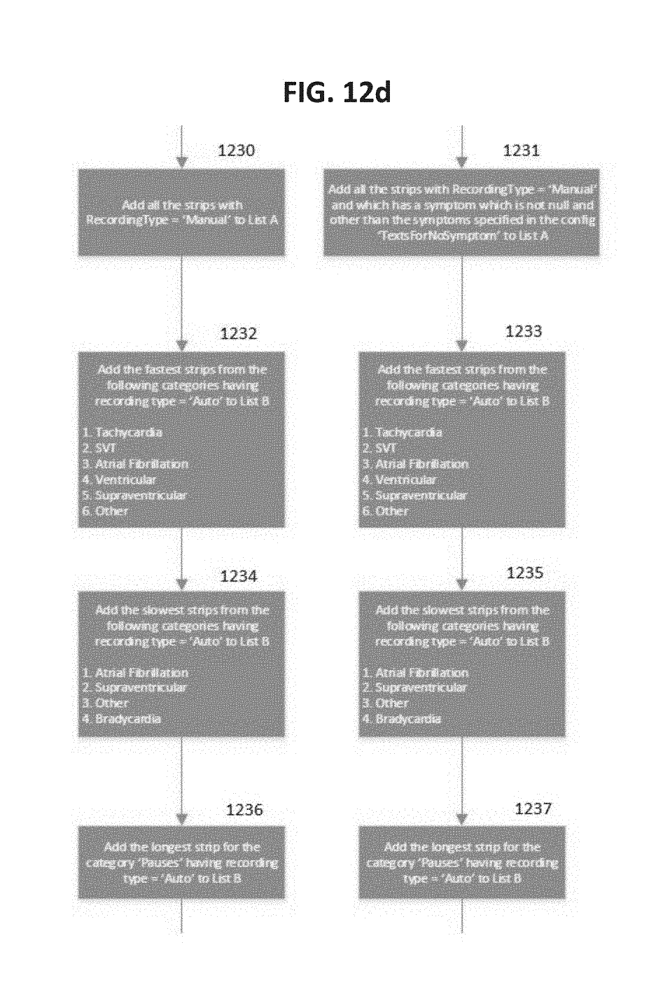

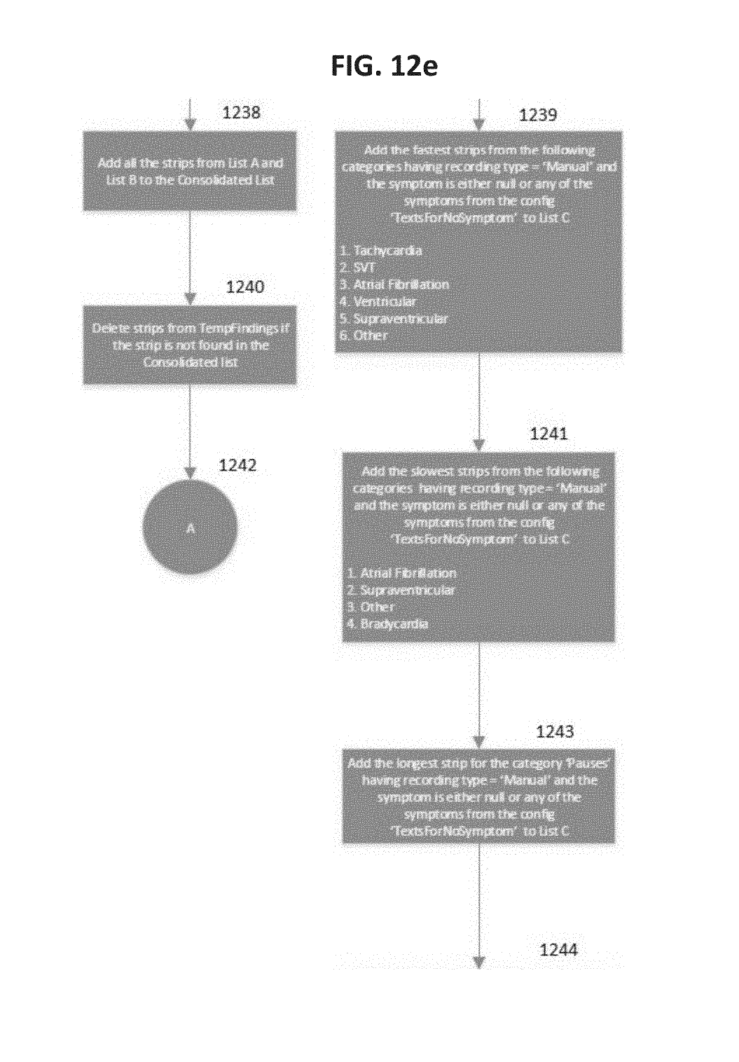

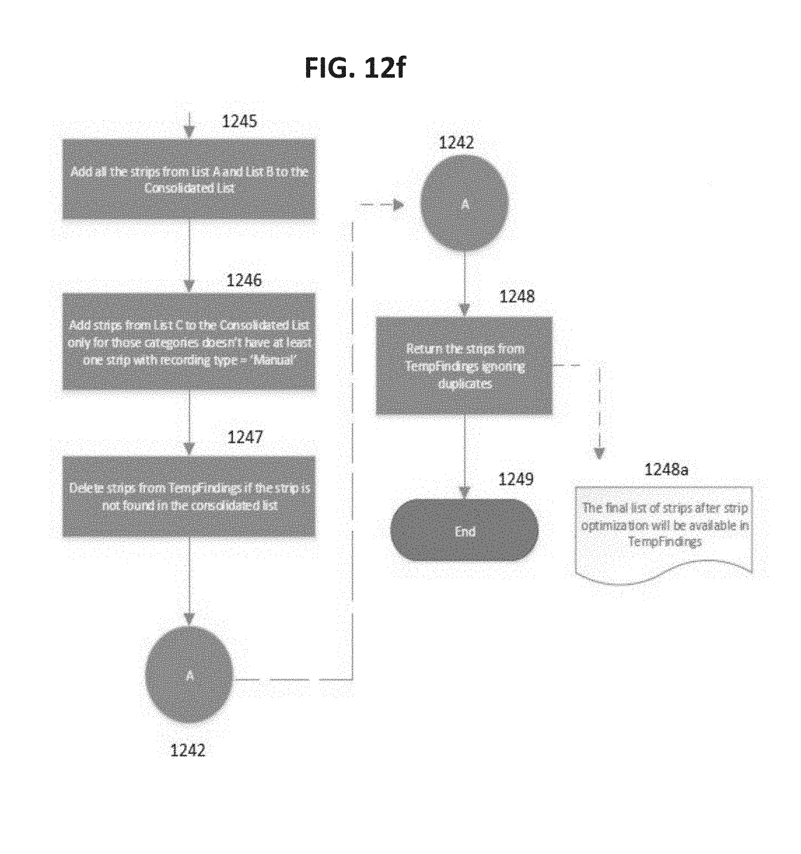

FIGS. 12a-12f are flowcharts illustrating an embodiment of a process of building a heart monitor graph with anomalous heart beat indication strips. Beginning with block 1202 of FIG. 12a, the variables that include the rules applied to fetching and displaying the reports are indicated. Block 1203 initializes the strip variables to zero and Boolean logic is applied to showing the marked strips. Block 1206 indicates that the report information is fetched. Block 1207 indicates that if no symptoms are presented, a display may indicate “no patient selection, no symptom selected, and/or symptom not specified by patient”. Block 1209 indicates that the display software 607 may create a list and insert strips corresponding to the current report which have recordings and the workflow status for recording. Boolean logic is applied to representative variables. Block 1210 indicates that all the steps that follow in FIGS. 12b-12f will be fetches from the list in block 1209. For daily reports, only the strips from that report are fetched. The same is true for weekly reports. For PTD and Summary reports all strips from the procedure are fetched.

FIG. 12b continues the flowchart by checking to see if the variables have content. If yes, the flow chart continues. If not, the chart skips to “A” 1213 at the end. The next step 1214 checks to see if the type of strip is manual 1215 or if it is standard 1214. If it is not all manual, the chart skips to “A” 1216 at the end. If it is all manual, the chart continues to block 1218 where three separate lists for storing the strips are created. Those are List A, List B, and consolidated lists. The chart also continues with a parallel path where if the type of strip is standard, then four separate lists for storing the strips are created. Those lists are List A, List B, List C, and a consolidated list. Block 1219 indicates that in List A, strips are based on the general optimization rule without considering the recording type. Notification, importance and manual with symptoms are included.

List B indicates that strips are based on the general optimization rule for the recording type “Auto”. List C indicates that strips are based on the general optimization rule for the recording type “manual” and symptom is null.

FIG. 12c continues the dual path between all manual recordings, meaning the patient was responsible for pressing a button on the pendant 701, 602, and standard recordings, meaning the heart beat data was automatically recorded. Even block numbers represent manual recordings and odd block numbers represent standard recordings. However, both the odd and the even blocks are identical.

Blocks 1220, 1221 indicate that the fastest strips from the following categories will be added to list A: NSR, Tachycardia, SVT, atrial fibrillation, ventricular, supraventricular, other. As block 1221a indicates, “Fastest Strips” means that those strips have the highest heart rate.

Blocks 1222, 1223 indicates that the slowest strips from the following categories are added to List A: NSR, Atrial fibrillation, supraventricular, other, Bradycardia. Block 1223b indicates that “Slowest Strips” are those that have the lowest heart rate.

Blocks 1224, 1225 indicate that the longest strips from the following categories are added to List A: Bradycardia, Tachycardia, SVT, Ventricular. Block 1225c indicates that “Longest Strips” are those that have the longest length.

Blocks 1226, 1227 indicate that the two longest strips for the category “pauses” are added to List A. Blocks 1228, 1229 indicate that strips marked where notification has been met or are identified as important, are added to List A.

FIG. 12d continues the dual path between all manual recordings and standard recordings. The blocks in FIG. 12d are identical, with the exception of Blocks 1230 and 1231. For the manual recording side, Block 1230 indicates that all strips with the recording type “Manual” are added to List A at this point. Block 1231 indicates that all strips that meet certain conditions for this path are added to List A.

Blocks 1232, 1233 indicate that the fastest strips from the following categories having the recording type “Auto” are added to List B: Tachycardia, SVT, Atrial Fibrillation, Ventricular, Supraventricular, Other.

Blocks 1234, 1235 indicate that the slowest strips from the following categories having a recording type “Auto” are added to List B: Atrial Fibrillation, Supraventricular, Other, Bradycardia.

Blocks 1236, 1237 indicate that the longest strip for the category “Pauses” having a recording type “Auto” are added to List B.

FIG. 12e continues the dual path between all manual recordings and standard recordings. Block 1238 indicates on the manual recording path that the strips from List A and List B are added to the consolidated List. Block 1240 indicates that strips are deleted from the temporary variable if the strip is not found in the consolidated list. Block 1242 indicates that this path continues to “A”, 1242.

The other path continues with Block 1239. Block 1239 indicates that the fastest strips from the following categories with a recording type of manual and that meet certain conditions are added to List C: Tachycardia, SVT, Atrial Fibrillation, Ventricular, Supraventricular, Other. Block 1241 continues by indicating that the slowest strips from the following categories having the recording type “manual” and that meet certain conditions are added to List C: Atrial Fibrillation, Supraventricular, Other, Bradycardia.

Block 1243 indicates that the longest strip for the category “pauses” having a recording type “manual” and that meets certain conditions are added to List C.

FIG. 12f continues the path of the standard recordings with block 1245. Block 1245 indicates that all the strips from List A and List B are added to the Consolidated List. Block 1246 indicates that the strips from List C are added to the Consolidated List only for all those categories that doesn't have at least one strip with recording type “Manual”. Block 1247 indicates that strips are deleted from the temporary variable if the strip is not found in the consolidated list. The flowchart continues to “A” at 1242. From there, block 1248 indicates that the strips are returned from the temporary findings variable and duplicates are ignored. Block 1248a indicates that the final list of strips after strip optimization will be available in the temporary findings variable.

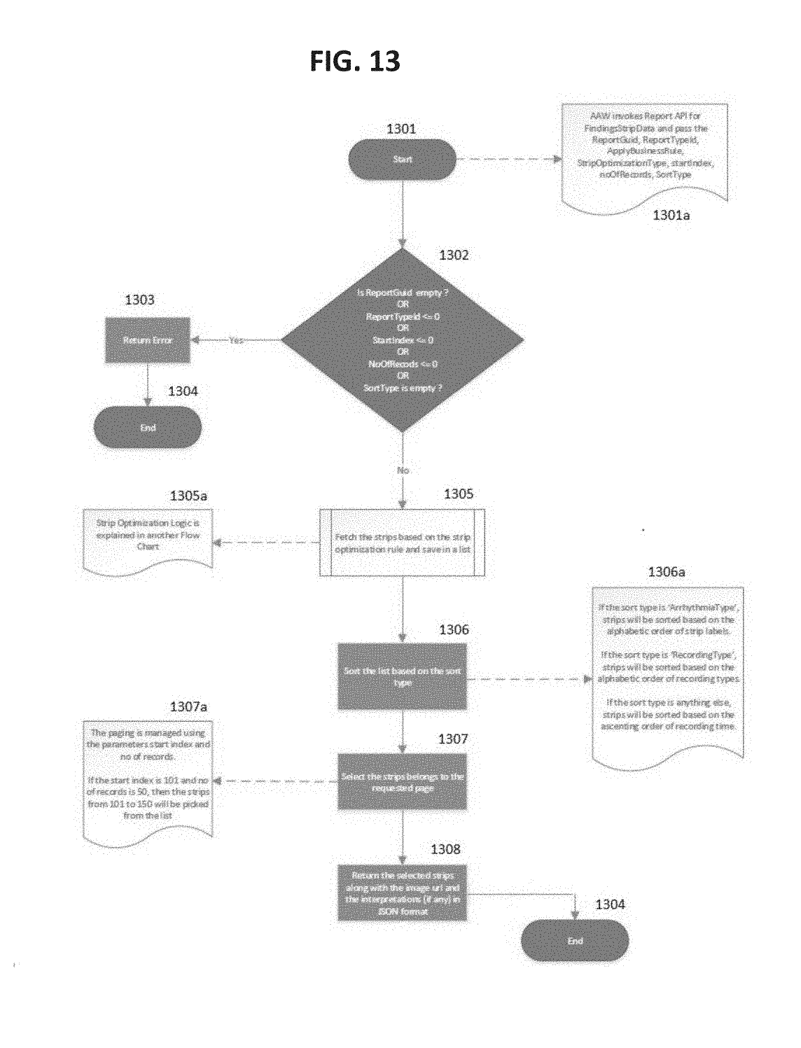

FIG. 13 is a flowchart illustrating one embodiment of how strip representations of heart beat activity are built. Block 1301a indicates that the display software 607 (AAW), invokes an API to pass along record information. Block 1302, 1303, and 1304 indicate error handling for the program. Block 1305 indicates that strips are fetched based on the optimization logic described in FIGS. 12a-12f. Block 1306 indicates that the list is sorted based on the sort type. Block 1306 explains that if the sort type is “Arrhythmia type” strips, the will be sorted based on the alphabetic order of strip labels. If the sort type is recording type, the strips will be sorted based on the alphabetic order of recording types. If the sort type is anything else, strips will be sorted based on the ascending order of recording time.

Block 1307 indicates that the strips are selected that belong to the requested page. As block 1307a indicates, the paging is managed using the certain parameters. Block 1308 indicates that the selected strips are then returned.

Some of the illustrative aspects of the present invention may be advantageous in solving the problems herein described and other problems not discussed which are discoverable by a skilled artisan.

While the above description contains much specificity, these should not be construed as limitations on the scope of any embodiment, but as exemplifications of the presented embodiments thereof. Many other ramifications and variations are possible within the teachings of the various embodiments. While the invention has been described with reference to exemplary embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best or only mode contemplated for carrying out this invention, but that the invention will include all embodiments falling within the scope of the appended claims. Also, in the drawings and the description, there have been disclosed exemplary embodiments of the invention and, although specific terms may have been employed, they are unless otherwise stated used in a generic and descriptive sense only and not for purposes of limitation, the scope of the invention therefore not being so limited. Moreover, the use of the terms first, second, etc. do not denote any order or importance, but rather the terms first, second, etc. are used to distinguish one element from another. Furthermore, the use of the terms a, an, etc. do not denote a limitation of quantity, but rather denote the presence of at least one of the referenced item. Thus, the scope of the invention should be determined by the appended claims and their legal equivalents, and not by the examples given.

That which is claimed is:

1. A heart monitor with diagnostic display comprising:
a plurality of electrodes configured to collect heart beat data from electrical pulses within a heart;
a processor adapted to receive the heart beat data from the plurality of electrodes and configured to connect with a long-range wireless network; and
a diagnostic display unit;
wherein the diagnostic display unit is configured to display biometric measurements comprising:
a first unit of time along a first axis;
a second unit of time, smaller than the first unit of time, along a second axis; and
a gradient block having a primary characteristic indicative of a primary characteristic of a cardiac event detected by the processor in the heart beat data during a collection time and located at an intersection of the first axis and the second axis corresponding to a collection time of cardiac activity.

2. The heart monitor with diagnostic display according to claim 1 wherein the second axis includes a dynamically adjustable scale.

3. The heart monitor with diagnostic display according to claim 1 wherein the gradient block has a secondary characteristic indicative of a secondary characteristic of the cardiac event.

4. The heart monitor with diagnostic display according to claim 1 wherein each of the plurality of gradient blocks is present only if a characteristic of the cardiac activity is above a threshold level.

5. The heart monitor with diagnostic display according to claim 1 wherein the collection time has a duration of 30 minutes.

6. The heart monitor with diagnostic display according to claim 1 wherein the primary characteristic of the gradient block is shade.

7. The heart monitor with diagnostic display according to claim 6 wherein a darker shade corresponds to a primary characteristic of a cardiac event of greater medical concern than a lighter shade.

8. The heart monitor with diagnostic display according to claim 1 wherein a primary characteristic of the cardiac event is frequency.

9. The heart monitor with diagnostic display according to claim 1 wherein the cardiac event comprises atrial fibrillation.

10. The heart monitor with diagnostic display according to claim 1 wherein the first unit of time comprises a calendar day;
wherein the first axis comprises the major axis;
wherein the second unit of time comprises the time of day; and
wherein the second axis comprises the minor axis.

11. A method of monitoring heart beat activity comprising:
utilizing a heart rate monitor to record electrical pulses output by a patient's heart, the heart rate monitor comprising a plurality of electrodes;
utilizing a portable computing device configured to receive data from the heart rate monitor and transmit the data via a long-range wireless network;
utilizing a receiving center to receive the data transmitted via the long-range wireless network;
transmitting the data received by the receiving center to at least one technician for the identification of anomalous heart beat data;
submitting the anomalous heart beat data into interface software;
utilizing the interface software to transmit the anomalous heart beat data to display software;
utilizing the display software to display, on a diagnostic display unit, the anomalous heart beat data;
wherein the diagnostic display unit is configured to display biometric measurements to a healthcare professional;
wherein the diagnostic display unit is configured to display:
a first unit of time along a first axis;
a second unit of time, smaller than the first unit of time, along a second axis; and
a plurality of gradient blocks each having a primary characteristic indicative of a primary characteristic of a cardiac event detected during a collection time and located at respective intersections of the first axis and second axis corresponding to respective collection times of cardiac activity; and
wherein each of the plurality of gradient blocks is present only if a characteristic of the cardiac activity is above a threshold level.

12. The method according to claim 11 wherein the second axis comprises a dynamically adjustable scale.

13. The method according to claim 11 wherein the plurality of gradient blocks comprises a secondary characteristic indicative of a secondary characteristic of the cardiac event.

14. The method according to claim 11 wherein the collection time comprises a duration of 30 minutes.

15. The method according to claim 11 wherein the primary characteristic of the plurality of gradient blocks is shade; and
wherein a primary characteristic of the cardiac event is frequency.

16. The method according to claim 15 wherein a darker shade corresponds to a greater frequency than a lighter shade.

17. The method according to claim 11 wherein the cardiac event comprises atrial fibrillation.

18. The method according to claim 11 wherein the first unit of time comprises a calendar day;
wherein the first axis comprises the major axis;
wherein the second unit of time comprises the time of day; and
wherein the second axis comprises the minor axis.

19. A method of monitoring heart beat activity comprising the steps of:
utilizing a heart monitor comprising:
a plurality of electrodes configured to collect heart beat data from electrical pulses within a heart;
a processor adapted to receive the heart beat data from the plurality of electrodes and configured to connect with a long-range wireless network; and
a diagnostic display unit;
wherein the diagnostic display unit is configured to display biometric measurements comprising:
a first unit of time along a first axis;
a second unit of time, smaller than the first unit of time, along a second axis; and
a gradient block having a primary characteristic indicative of a primary characteristic of a cardiac event detected by the processor in the heart beat data during a collection time and located at an intersection of the first axis and the second axis corresponding to a collection time of cardiac activity;
utilizing the processor to transmit the data via a long-range wireless network;
utilizing a receiving center to receive the data transmitted via a long-range wireless network;
transmitting the data from the receiving center to at least one technician for the identification of anomalous heart beat data;
submitting the anomalous heart beat data into interface software;
utilizing the interface software to transmit the anomalous heart beat data to display software within the pendant;
utilizing the display software to display on the pendant the anomalous heart beat data; and
wherein the pendant s configured to display biometric measurements to a healthcare professional.

20. The method according to claim 19 wherein each of the plurality of gradient blocks is present only if a characteristic of the cardiac activity is above a threshold level.
